(19) United States
(12) Patent Application Publication
(10) Pub. No.: US 2020/0093522 A1
(43) Pub. Date: Mar. 26, 2020

(54) Device for Extraction of Pedicle Screw

(71) Applicants: Thomas Zink, San Antonio, TX (US); Zeshan Hyder, Munster, IN (US)

(72) Inventors: Thomas Zink, San Antonio, TX (US); Zeshan Hyder, Munster, IN (US)

(21) Appl. No.: 16/572,691

(22) Filed: Sep. 17, 2019

Related U.S. Application Data
(60) Provisional application No. 62/734,316, filed on Sep. 21, 2018.

(51) Int. Cl. A61B 17/70

(52) U.S. Cl. CPC A61B 17/7091

(57) ABSTRACT

A pedicle screw extraction device that comprises a cannulated shaft and an attached pedicle screw acceptance component secured on the distal end of the shaft, as well as a rod position in and extending outwardly from the distal and proximal ends of the cannulated shaft. The pedicle screw acceptance component comprises a tapered reverse thread for engaging a pedicle screw, while the rod prevents the head of the pedicle screw from rotating during the extraction process. The shaft will have a handle adapter that allows for the attachment of various types and sizes of handles as well as a lateral extrusion on the proximal portion that allows for the maximizing of torque during rotation of the shaft and the attached pedicle screw.

### CROSS-REFERENCE TO RELATED APPLICATION

The present application claims priority from U.S. Provisional Patent Application Ser. No. 62/734,316 filed on Sep. 21, 2018, which is incorporated herein by reference.

### FIELD OF THE INVENTION

The present invention relates generally to a pedicle screw extraction device and, more particularly, to a pedicle screw extraction device that: (i) eliminates the need for multiple extraction devices and/or attachments therefore to accommodate different pedicle screw sizes and/or configurations that may be encountered during the extraction process; (ii) significantly reduces the likelihood that the pedicle screw will need to be cut or broken during the extraction process; and (iii) is particularly useful for removing stripped or overgrown pedicle screws from a patent.

### BACKGROUND

A number of medical conditions exist, such as compression of spinal cord nerve roots, degenerative disc disease, tumors, and trauma that can cause severe back pain and patient discomfort. Intervertebral fusion is one surgical method commonly employed to alleviate back pain and discomfort. In an intervertebral fusion procedure, two adjacent vertebral bodies are fused together by removing the affected or damaged intervertebral disc, and replacing it with an implant that would allow for bone to grow between the two vertebral bodies to bridge the gap left by the removed disc.

Another common surgical method of relieving back pain is a corpectomy procedure. In a corpectomy procedure, a diseased or damaged vertebral body and the adjoining intervertebral discs are removed (usually as a means of decompressing the spinal cord) and replaced with a spinal implant that would allow for bone to grow between the closest two adjacent vertebral bodies to bridge the gap left by the removed spinal tissue and discs.

Heretofore, a number of different implant materials and implant designs have been used in intervertebral fusion and for vertebral body replacement with varying degrees of success. Current implant materials used include metals, radiolucent materials including plastics, elastic and polymeric materials, ceramic, and allografts. Current implant designs vary from threaded cylindrical implants to rectangular cages with teeth-like protrusions to better grip the adjacent vertebra.

Further, the pedicle screw is another tool that a spine surgeon will oftentimes use in helping to treat various spinal and lower back problems. More specifically, the pedicle screw is used in conjunction with spinal fusion surgery to further fixate the vertebrae in order to inhibit their movement during the fusion and/or healing process. The effectiveness of the pedicle screw is inherently related to its precise placement in the body, and therefore it is less effective if not properly positioned. The pedicle screw, however, is not a solution on its own, but rather a means of providing stability to the spine and fixating the bone graft as the fusion process takes place. Because of its nature as a support or stabilizing device, once the bone graft and the adjacent vertebrae have successfully fused, the pedicle screw is no longer medically necessary and is preferably removed from the patient. This is true because while a large percentage of patients do not require the removal of the pedicle screw, the continued implantation of the screw can cause discomfort for the patient.

Heretofore, various systems, instruments and methods have been employed by surgeons to remove pedicle screws from patients when the same are no longer required. Unfortunately, many different removal devices and attachments therefore are required to be kept readily available given the various pedicle screw insertion features, sizes and configurations that may be encountered during a pedicle screw extraction, and the acquisition and storage of the same could be both expensive and inefficient. Further, after existing in vivo for an extended period of time, pedicle screws can become filled with organic material and/or stripped during the installation or removal processes, thereby making their successful removal difficult. In fact, oftentimes, filled or damaged pedicle screws must be cut or severed and then removed utilizing the remaining portions of the pedicle screw.

As previously stated, another issue associated with prior art pedicle screw removal instruments is the cost associated with having to obtain and maintain many different types of pedicle screw removal devices and/or bits to accommodate the various sizes, shapes and configurations of pedicle screws that may be encountered. These removal devices and/or bits are not only expensive, but may also become stripped over time or lost in the surgical process, thereby necessitating their replacement which can also be both costly and inconvenient. Further, the heads of adjacent pedicle screws may be in contact with the pedicle screw to be removed, therefore making the cutting of the rod between screw heads impossible as a removal technique.

Consequently, there is a long felt need in the art for a pedicle screw extraction device that allows for the removal of pedicle screws without the need to purchase and maintain many different and differently sized extraction devices and/or attachments therefore. There is also a long felt need in the art for a pedicle screw extraction device that enables the surgeon to easily remove a pedicle screw without the need to cut or break the pedicle screw. Finally, there is also a long felt need in the art for a pedicle screw extraction device that is relatively easy to use, inexpensive to manufacture and that works with all types and sizes of pedicle screws, rod sizes, patient anatomy, and surgical approaches.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

In a preferred embodiment of present invention, the pedicle screw extraction device comprises an elongated shaft having a continuous opening therein, a rod movably and rotatably positioned within said opening in the shaft, a handle positioned on a first or proximal end of said rod and a t-shaped component positioned on a second end of said rod, a torque amplification device positioned on a proximal end of the elongated shaft and a pedicle screw acceptance component positioned on a distal end of the elongated shaft. The pedicle screw acceptance component could be integral to the elongated shaft, or it could be attached thereto as explained more fully below.

In an alternative embodiment of the present invention, the distal portion of the elongated shaft prevents extended movement of the acceptance component after the acceptance component engages the pedicle screw head to secure to pedicle screw to the device, and the range of motion of the head of the pedicle screw is inhibited by the rod. By way of background, a pedicle screw is typically comprised of a head and a threaded shaft, wherein the head of the pedicle screw is typically rotatable about a proximal end of the pedicle screw shaft.

In yet another embodiment of the present invention, an exterior surface of the distal end of the elongated cannulated shaft and/or the acceptance component are threaded with a tapered reverse thread for engaging the head of a pedicle screw. In one embodiment, the distal portion of the shaft may comprise a threaded opening and the acceptance component may comprise an extruded thread that is accepted by the threaded opening of the shaft such that the two bodies are concentrically formed. In an alternative embodiment, the acceptance component may further comprise a generally circular rig having a diameter that is greater than the diameter of the elongated shaft, such that the circular rig can be rotated to thread the acceptance component in and out of the threaded shaft.

In yet another embodiment of the present invention, after threading of the acceptance component to the shaft, the acceptance component and/or rod can move freely in the vertical direction as well as be rotated around its longitudinal axis such that it can adapt to the position of the pedicle screw head in the body. In a further preferred embodiment, the acceptance component will be able to rotate around its longitudinal axis independent of the threading such that the engagement mechanism can be manually aligned with the position of the pedicle screw head in the patient. In an even further preferred embodiment, the acceptance component will rotate on the same axis as the rod such that the handle associated with the rod may be used to align the acceptance component with the pedicle screw head.

In a further embodiment, the device further comprises a cage on the distal end of the elongated shaft with an opening therein for receipt of the pedicle screw head. In this embodiment, the bottom of the cage will be of a diameter less than the pedicle screw head, but greater than the body of the pedicle screw such that the device grasps onto the pedicle screw despite the head not being completely fixated yet. In a preferred embodiment, the width of the opening will be great enough to negate the need for the user to employ their hands to align the opening and the head of the pedicle screw, but still allow for the engagement of the screw by the cage.

In yet another embodiment, the device of the present invention further comprises an acceptance component comprised of a T-shaped extrusion on the distal end that is adapted to the shape of the pedicle screw head such that the acceptance component engages the pedicle screw and fixates it while it is further secured through the rotation of the circular rig until the acceptance component is fully threaded onto the shaft.

In another embodiment, the elongated shaft may be comprised of a lateral extrusion on its proximal end. The lateral extrusion may be of such a length and diameter that enables the user to apply external longitudinal force, and amplify the torque such that the pedicle screw can be more easily extracted from the patient. In a further preferred embodiment, the lateral extrusion is positioned in a vertical position such that torque is maximized, and the lateral extrusion is shaped to increase user accessibility and ease of use.

In yet another embodiment, the elongated shaft may be further comprised of a screw acceptance component with which it is integrally formed. The pedicle screw acceptance component may comprise a tapered reverse thread and a rod on the distal portion that holds the head of the pedicle screw in place, and prevents it from spinning when the shaft is rotated in an opposite direction to the threading of the pedicle screw acceptance component. Thus, the pedicle screw acceptance component remains engaged with the pedicle screw while the pedicle screw is extracted from the patient.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

While the specification concludes with claims particularly pointing out and distinctly claiming the present invention, it is believed that the present invention will be better understood from the following description in conjunction with the accompanying FIGS., in which like reference numerals identify like elements, and wherein:

FIG. 1 illustrates a front perspective view of one embodiment of the pedicle screw extraction device of the present invention engaged with a pedicle screw in accordance with the disclosed architecture.

FIG. 2 illustrates a side perspective view of the pedicle screw extraction device of FIG. 1 engaged with a pedicle screw in accordance with the disclosed architecture.

FIG. 3 illustrates an auxiliary view of the distal end of the pedicle screw extraction device engaged with a pedicle screw in accordance with the disclosed architecture.

FIG. 4 illustrates an angled top view of a portion of a pedicle screw in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The present invention relates to a uniquely structured pedicle screw extraction device comprising an acceptance component and a hollow shaft that is machined, reverse threaded, and removably attached to, or integrally formed with, the acceptance component. Further, the pedicle screw extraction device provides an easy and efficient process for removing implanted pedicle screws of any shape, size, or position.

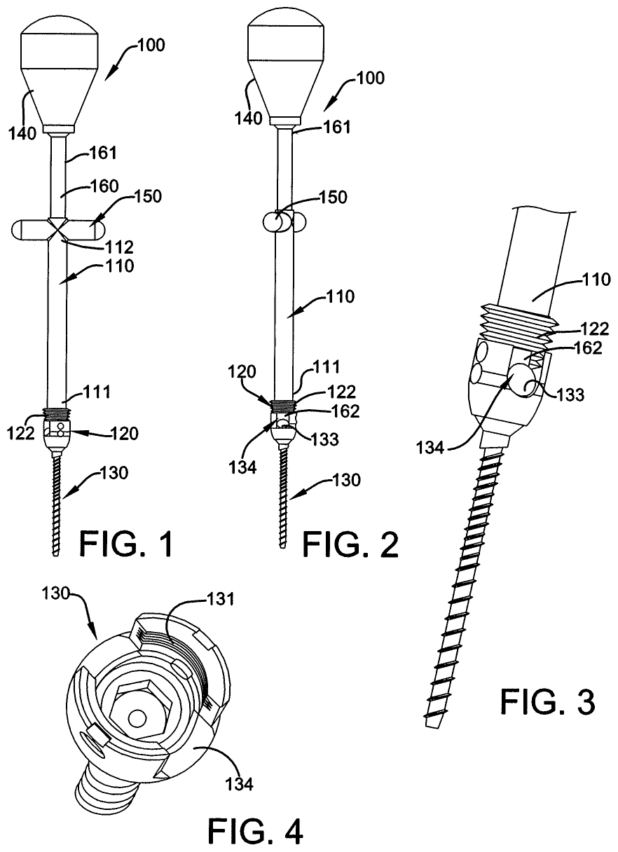

Referring initially to the drawings, FIG. 1 illustrates a perspective view of one embodiment of a pedicle screw extraction device 100 of the present invention. The pedicle screw extraction device 100 is preferably comprised of an elongated shaft 110 having a continuous opening therein (not shown), a pedicle screw acceptance component 120 for securing the shaft 110 to a pedicle screw 130, a handle component 140, a torque amplification component 150, and a rod 160 movably and rotatably positioned within said opening in shaft 110.

Shaft 110 is preferably comprised of a distal end 111 and a proximal end 112 and is typically a cannulated cylindrical tube. As best shown in FIGS. 1-2, the surfaces of each of the proximal 112 and distal 111 ends, where other mechanisms or apparatuses are not present, are typically relatively smooth, but do not have to be. More specifically, the relatively smooth surfaces of distal end 111 and proximal end 112 allows for easy gripping of the shaft 110 by the user. Additionally, the surfaces of the shaft 110 can be textured or ribbed to provide more gripping and reduce slippage by the user.

Notwithstanding the foregoing, one of ordinary skill in the art will appreciate that the shape and size of the shaft 110, as shown in FIGS. 1-3, is for illustrative purposes only and that many other shapes and sizes of shaft 110 are well within the scope of the present disclosure. Although dimensions of shaft 110 (i.e., length, width, and height) are important design parameters for good performance, shaft 110 may be any shape or size that ensures optimal performance during use and is within the overall objective of the present invention.

Additionally, the shaft 110 is typically manufactured from stainless steel, but can be manufactured from any other suitable material as is known in the art for sterile or surgical applications. Further, shaft 110 may be manufactured using machining techniques, or by using a combination of other molding or machining techniques (additive manufacturing, etc.). These additional techniques include, without limitation, material extrusion, vat photo polymerization, powder bed fusion, material jetting, binder jetting, sheet lamination and directed energy deposition.

As best shown in FIG. 3, pedicle screw acceptance component 120 is positioned at the distal end 111 of the shaft 110 and is used for engagement of the pedicle screw 130. More specifically, the pedicle screw acceptance component 120 comprises a tapered reverse thread 122 that will be of such an angle and width of extrusion as to allow for acceptance of a wide array of various sizes and orientations of pedicle screws 130. The reversed orientation of the tapered reverse thread 122 will allow for the removal of the pedicle screw 130 from the body while still allowing for the attachment of the pedicle screw acceptance component 120 to the pedicle screw 130. Of course, one of ordinary skill in the art will appreciate that many other angled configurations/combinations may also be used to enable the device 100 to securely engage the pedicle screw 130 as is known in the art.

As previously stated, the pedicle screw extraction device 100 of the present invention further comprises a rod 160 positioned at least partially within the cannulated shaft 110 and extending outwardly from both the proximal end 112 and the distal end 111 of the shaft 110, and past the tapered reverse thread 122. In one embodiment, the rod 160 is secured within the shaft 110 via threading the rod 160 to the proximal end 112 of the shaft 110. Nonetheless, if not secured, the rod 160 is also capable of moving longitudinally and rotatably within shaft 110 via a pin connection. Rod 160 is further comprised of a first or proximal end 161 with handle 140 positioned thereon, and a second or distal end 162, with a generally t-shaped component 133 positioned thereon for matingly engaging pedicle screw 130, as best shown in FIG. 3. More specifically, when the pedicle screw 130 is secured to the tapered reverse thread 122, the rod 160 may be pressed against the head of the pedicle screw 130 so that the generally t-shaped component 133 engages a slot 134 in the head of the pedicle screw 130, as best shown in FIG. 4. In this manner, the rod 160 prevents the head of the pedicle screw 130 from unwanted rotation, unless the user rotates rod 160 within shaft 110 to intentionally rotate pedicle screw 130. Nonetheless, it is contemplated that component 133 could be configured differently (i.e., so that it isn't generally T-shaped), but still successfully engages the head of pedicle screw 130.

As best shown in FIG. 1-2, handle 140 allows for easy handling of device 100, and for longitudinally and rotatably repositioning rod 160, as desired. Handle 140 is preferably conically shaped for easy and ergonomical handling, but one of ordinary skill in the art will appreciate that the shape and size of the handle 140 are for illustrative purposes only, and that many other shapes and sizes of handle 140 are well within the scope of the present disclosure. Further, the handle 140 can be secured to the rod 160 via any attachment means as is known in the art (e.g., threading, etc.) or may be integrally formed or manufactured. As previously mentioned, the pedicle screw acceptance component 120 and the elongated shaft 110 may also be manufactured together as a single integral component, or may be manufactured separately and then integrally attached in accordance with the desired wants and/or needs of a user.

Additionally, the shaft 110 comprises at least one lateral extrusion or torque amplification component 150. Specifically, the shaft 110 depicted in FIGS. 1 and 2 comprises two lateral extrusions or torque amplification components 150 which extend outwardly therefrom and that are preferably positioned on the proximal end 112 of the shaft 110. The lateral extrusions or torque amplification components 150 are secured to the shaft 110 via any suitable securing method or means as is known in the art for securing two objects, such as metal to metal. The lateral extrusions or torque amplification components 150 allow for the amplification of torque by the surgeon or other user when rotating the elongated shaft 110 to assist in the removal of the pedicle screw 130 from the patient.

During surgical removal of pedicle screw 130 from a patient (not shown), pedicle screw acceptance component 120 engages pedicle screw 130 by screwing the tapered reverse thread 122 into the accepting thread 131 of the pedicle screw 130 as the rod prevents the head of the pedicle screw 130 from rotating. Once the pedicle screw acceptance device 120 and the pedicle screw 130 are properly fastened together, the shaft 110 is rotated in the direction counter to that used to screw the tapered reverse thread 122 into the accepting thread 131 so that the shaft 110 and the pedicle screw 130 remained fastened while allowing for the extraction of the pedicle screw 131 from the patient. After the pedicle screw 130 has been successfully extracted from the patient, it can be disengaged from the pedicle screw acceptance component 120 by unscrewing the tapered reverse thread 122 from the acceptance thread 131.

Additionally, as the pedicle screw acceptance component 120 of elongated shaft 110 is threaded into the pedicle screw 130, rotation and counter-rotation of the head of the pedicle screw 130 is prevented by rod 160. More specifically, rod 160 engages the head of the pedicle screw 130 (i.e., via inserting t-shaped component 133 into slot 134), and prevents the same from rotating relative to the body of threaded shaft of pedicle screw 130, which, in turn, allows a user to rotate the pedicle screw extraction device 100 which allows for extraction of the pedicle screw 130 from the patient.

In an alternative embodiment, the distal end 111 of the shaft 110 may comprise a threaded opening (not shown) and the acceptance component 120 may comprise an extruded thread (not shown) that is accepted by the threaded opening of the shaft 110 such that the two bodies are concentrically formed. In an even further alternative embodiment, the acceptance component 120 comprises a circular rig (not shown) having a diameter greater than the diameter of the shaft 110, such that the circular rig can be rotated to thread the acceptance component 120 in and out of the threaded elongated shaft 110.

In a further alternative embodiment, the device 100 further comprises a cage (not shown) on the distal end 111 of the elongated shaft 110 with an opening therein (not shown) for receipt of the head of pedicle screw 130. In this embodiment, the bottom of the cage will be of a diameter less than the head of pedicle screw 130, but greater than the body of the pedicle screw 130 such that the device 100 grasps onto the pedicle screw 130 despite the head not being completely fixated yet. In a preferred embodiment, the width of the opening will be great enough to negate the need for the user to employ his or her hands to align the opening and the head of the pedicle screw 130, but still allow for the engagement of the pedicle screw 130 by the cage.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A pedicle screw extraction device comprising:
an elongated shaft comprising a continuous opening therein, a distal end and a proximal end;
a rod at least partially positioned in the continuous opening;
a torque amplification device; and
a pedicle screw acceptance component.

2. The pedicle screw extraction device of claim 1, wherein the rod further comprises a handle portion positioned on a first end of said rod and a t-shaped component positioned on a second end of said rod.

3. The pedicle screw extraction device of claim 1, wherein the torque amplification device is positioned on the proximal end of the elongated shaft.

4. The pedicle screw extraction device of claim 1, wherein the rod is movably positioned within the elongated shaft.

5. The pedicle screw extraction device of claim 1, wherein the rod is rotatably positioned within the elongated shaft.

6. The pedicle screw extraction device of claim 1, wherein the distal end of the elongated shaft is further comprised of reverse threads.

7. A pedicle screw extraction device comprising:
a cannulated shaft comprising a distal end and a proximal end;
a rod at least partially positioned within the cannulated shaft;
a pedicle screw acceptance component secured on the distal end; and
a torque amplification device positioned on the proximal end.

8. The device of claim 7, wherein the rod comprises a t-shaped component that engages a pedicle screw and prevents movement of the pedicle screw during extraction of the pedicle screw.

9. The device of claim 8, wherein the pedicle screw acceptance component is a threaded opening which secures to a head of the pedicle screw.

10. The device of claim 7, wherein the cannulated shaft comprises at least one lateral extrusion positioned on the proximal end.

11. The device of claim 7 further, wherein the cannulated shaft comprises two lateral extrusions on the proximal end of the cannulated shaft.

12. A pedicle screw extraction device comprising:
a cannulated shaft comprising a distal end and a proximal end; and
a pedicle screw acceptance component secured to the distal end; and
wherein the pedicle screw acceptance component is comprised of a reversed tapered thread.

13. The device of claim 12 further comprising a rod movably and rotatably positioned at least partially within the cannulated shaft.

14. The device of claim 12, wherein the cannulated shaft comprises two lateral extrusions which extend outwardly from the proximal end of the cannulated shaft.

15. The device of claim 13, wherein the rod is further comprised of a first end with a handle positioned thereon and a second end with a t-shaped component thereon for matingly engaging a head of a pedicle screw.

16. The device of claim 13 further comprising a torque amplification component for rotating the cannulated shaft about the rod.

17. The device of claim 13, wherein the cannulated shaft further comprises a longitudinal axis and the rod is slidable along said longitudinal axis relative to the cannulated shaft.

18. The device of claim 12, wherein the device is manufactured of stainless steel.
